(19) United States
(12) Patent Application Publication
(10) Pub. No.: US 2009/0156946 A1
(43) Pub. Date: Jun. 18, 2009

(54) BLOOD PRESSURE MOTION SENSING

(75) Inventors: John A. Lane, Weedsport, NY (US); Tyson B. Whitaker, Arden, NC (US); Matthew J. Kinsley, Liverpool, NY (US); Scott A. Martin, Warners, NY (US); Thomas J. Grant, Skaneateles, NY (US); Stephen F. Meyer, Skaneateles, NY (US)

Correspondence Address: Hiscock & Barclay, LLP, One Park Place, 300 South State Street, Syracuse, NY, 13202-2078, US

(73) Assignee: Welch Allyn, Inc., Skaneateles Falls, NY (US)

(21) Appl. No.: 11/955,823

(22) Filed: Dec. 13, 2007

(51) Int. Cl. A61B 5/022

(52) U.S. Cl. 600/490

(57) ABSTRACT

The invention relates to a blood pressure monitor for measuring the blood pressure of a person including a blood pressure cuff. The blood pressure monitor also includes an electro-pneumatic package. The electro-pneumatic package includes a pump, a valve, a pressure sensor, and one or more accelerometers. A pneumatic mechanical coupling is configured to pneumatically and mechanically directly couple the blood pressure cuff to the electro-pneumatic package, wherein a signal from the one or more accelerometers configured to indicate an activity level of the person during a blood pressure measurement. A display is configured to display an indication of the activity level. The invention also relates to a blood pressure monitor wherein an electronics circuit is configured to receive the motion signal from the one or more accelerometers during a blood pressure measurement. The invention also relates to a method for detecting a motion artifact in a non-invasive blood pressure measurement.

### FIELD OF THE INVENTION

This invention relates generally to blood pressure monitoring and more particularly to blood pressure monitoring apparatus including motion artifact sensing.

### BACKGROUND OF THE INVENTION

Non-invasive blood pressure (NIBP) monitors typically use a blood pressure cuff. To measure a person's blood pressure, a blood pressure cuff of an appropriate size can be affixed to a person's limb, typically to the upper portion of an adult's arm or to an infant's leg. The blood pressure cuff generally includes at least one inflatable bladder. Blood pressure measurements are made by inflating the bladder and then monitoring the pressure of the bladder using a pressure sensor as the bladder is deflated. Because the person's heart is pumping blood through arteries in the limb constrained by the blood pressure cuff, the bladder pressure measured by the pressure sensor includes pressure changes caused by pumping blood superimposed on the changing bladder pressure.

Systolic pressure is the maximum arterial pressure during contraction of the left ventricle of the heart. Diastolic pressure is the minimum arterial pressure during relaxation and dilatation of the ventricles of the heart when the ventricles fill with blood. Systolic pressure and diastolic pressure readings are two of the blood pressure parameters that a non-invasive blood pressure monitor can measure. Using the well known oscillometric method, the blood pressure cuff is initially inflated to a pressure higher than the highest expected systolic reading and then deflated to a pressure lower than the lowest expected diastolic reading. Above the systolic pressure, the pressure sensor signal reflects only the dominant cuff pressure. When the bladder pressure falls to a pressure near the systolic pressure, the pressure sensor signal begins to indicate cuff pressure oscillations superimposed on the deflating cuff pressure signal. When the cuff bladder pressure falls below the lower diastolic pressure, the cuff pressure oscillations are no longer present. An additional parameter, mean arterial pressure (MAP), can also be measured using the oscillometric method by further analysis of the cuff pressure oscillations that occur between the systolic pressure and diastolic pressure.

Most types of blood pressure monitors can produce reasonably accurate readings if the person's limb is substantially motionless during the deflation of the blood pressure cuff bladder. However, if the limb to which the blood pressure cuff is affixed is in motion during the blood pressure cuff bladder deflation, the pressure sensor can respond to additional motion induced pressure artifacts. In addition to conscious movement of the limb, motion artifact can be caused by involuntary motion such as can be caused by shivering and tremors. In many cases the magnitude of the motion can cause significant error in the blood pressure measurement results. For example, we have observed persons in a hospital setting where motion of limbs caused repeated errors in blood pressure measurements. In some cases, particularly where the NIBP measurement equipment is fully automatic, a clinician might be distracted and not realize that a blood pressure measurement has been corrupted by excessive patient motion. NIBP motion artifact can also be problematic during patient vehicular transport motion.

One solution to the motion artifact problem in NIBP monitors has been implemented under the trade name Smartcuf® technology by the Welch Allyn Corporation. The Smartcuf technology combines NIBP with ECG information. The Smartcuf technology can identify and disregard as attributable to motion artifact, pressure oscillations that occur at incorrect times with respect to heart pumping as monitored by the ECG. NIBP-ECG motion artifact detection and correction can be very effective, as indicated in the 1998 Revision Labs study, “Noninvasive Blood Pressure Measurement and Motion Artifact: A Comparative Study.” The NIBP-ECG motion artifact solution, however, is most cost effective when associated with a multifunction medical monitor already including both NIBP and ECG measurements. The problem is that ECG measurement signals are not typically available in the context of many stand alone NIBP clinical monitors as well as in most home use applications. Also, NIBP-ECG motion artifact detection and correction is cost prohibitive in the context of low cost NIBP single function instruments.

Another solution to the motion artifact problem in NIBP monitors studied by the Welch Allyn Corporation was described in U.S. patent application Ser. No. 10/619,380, “Motion management in a fast blood pressure measurement device” published as U.S. Published Patent Application No. 2005/0033188. In this solution, motion artifact was detected by analyzing a pressure signal indicative of the pressure of a bladder in a blood pressure cuff. The '380 application is incorporated herein by reference in its entirety.

What is needed is a blood pressure monitor that can better indicate when a blood pressure measurement has been corrupted by motion artifact. What is also needed is a blood pressure monitor that can correct blood pressure readings by removing the effects of motion artifact.

### SUMMARY OF THE INVENTION

In one aspect, the invention relates to a blood pressure monitor for measuring the blood pressure of a person including a blood pressure cuff. The blood pressure cuff includes at least one bladder. The blood pressure monitor also includes an electro-pneumatic package. The electro-pneumatic package includes a pump, a valve, a pressure sensor, and one or more accelerometers. The blood pressure monitor also includes a pneumatic mechanical coupling. The pneumatic mechanical coupling is configured to pneumatically and mechanically directly couple the blood pressure cuff to the electro-pneumatic package, wherein a signal from the one or more accelerometers configured to indicate an activity level of the person during a blood pressure measurement. The blood pressure monitor also includes a display. The display is configured to display an indication of the activity level.

In one embodiment the signal from the one or more accelerometers indicates an activity level of the person during a blood pressure measurement and an algorithm running on a microcomputer is configured to receive one or more values representative of the signal from the one or more accelerometers and the algorithm rejects at least one pressure value representative of a pressure signal from the pressure sensor as cause to be in error by the activity level of the person.

In another embodiment, the signal from the one or more accelerometers indicates an activity level of the person during a blood pressure measurement and an algorithm running on a microcomputer is configured to receive one or more values representative of the signal from the one or more accelerometers and the algorithm corrects at least one pressure value representative of a pressure signal from the pressure sensor to substantially remove motion induced error caused by the activity level of the person.

In yet another embodiment, the signal from the one or more accelerometers indicates an activity level of the person during a blood pressure measurement, the activity level of the person causing a motion induced error, and an algorithm running on a microcomputer is configured to receive one or more values representative of the signal from the one or more accelerometers and the algorithm corrects the blood pressure of the person to substantially remove the motion induced error.

In another aspect, the invention relates to a blood pressure monitor including a blood pressure cuff including at least one bladder. The blood pressure cuff also includes a first half of a mechanical pneumatic connector. The blood pressure monitor also includes a pump. The pump is pneumatically coupled to the bladder and configured to inflate the bladder. The blood pressure monitor also includes a valve. The valve is pneumatically coupled to the bladder and configured to cause a controlled deflation of the bladder. The blood pressure monitor also includes a pressure sensor. The pressure sensor is pneumatically coupled to the bladder and configured to measure a bladder pressure. The blood pressure monitor also includes one or more accelerometers. The one or more accelerometers are electrically and mechanically configured to provide a motion signal from the one or more accelerometers responsive to and representative of a movement of the at least one blood pressure inflatable bladder. The blood pressure monitor also includes an electronics circuit. The electronics circuit is electronically coupled to the valve, the pump, the pressure sensor, and the one or more accelerometers, wherein the pump, the valve, the pressure sensor, the one or more accelerometers, and the electronics circuit are disposed within an electro-pneumatic package, and the electro-pneumatic package include a second half of a mechanical pneumatic connector and wherein the first half of the mechanical pneumatic connector is configured to mechanically connect to the second half of a mechanical pneumatic connector to provide a substantially air-tight semi-rigid mechanical and pneumatic direct coupling between the electro-pneumatic package and the blood pressure cuff and wherein the electronics circuit is configured to receive the motion signal from the one or more accelerometers during a blood pressure measurement.

In one embodiment, the electronics circuit is configured to cause an indication of excessive motion based on the motion signal from the one or more accelerometers representative of the movement of the at least one bladder when the motion signal exceeds a pre-determined threshold.

In another embodiment, the indication of excessive motion includes one of a visual indication and an audio indication.

In yet another embodiment, the indication of excessive motion is configured to indicate that the blood pressure measurement should be repeated.

In yet another embodiment, the electronics circuit further comprises a microcomputer, the microcomputer configured to receive the motion signal from the one or more accelerometers and to correct the blood pressure measurement based on the motion signal.

In yet another embodiment, the one or more accelerometers comprise a MEMS accelerometer.

In yet another embodiment, the MEMS accelerometer includes a three axis accelerometer.

In yet another embodiment, the at least one accelerometer is mechanically disposed on or in a blood pressure monitor housing.

In yet another embodiment, the motion signal includes one or more analog signals from the one or more accelerometers and wherein the indication of excessive motion is based at least in part upon the motion signal and the motion signal threshold.

In yet another embodiment, the motion signal includes one or more analog signals from the one or more accelerometers and the electronics circuit includes at least one analog to digital converter (ADC), the at least one ADC configured to digitize the one or more analog signals from the one or more accelerometers, and wherein the indication of excessive motion is based at least in part upon the motion signal in a digital form and the motion signal threshold in a digital form.

In yet another embodiment, the electronics circuit is additionally configured to provide a correction to substantially correct the blood pressure measurement for a blood pressure cuff motion induced error.

In yet another embodiment, the correction to the blood pressure measurement includes a correction based on analog signals or a correction based on digital signals.

In yet another embodiment, the correction is configured to be applied to a digital representation of a pressure signal from the pressure sensor.

In yet another embodiment, the correction is configured to be applied as part of a digital computation used to calculate the blood pressure measurement.

In another aspect, the invention features a method for detecting a motion artifact in a non-invasive blood pressure measurement comprising the steps of: providing a blood pressure cuff having at least one bladder, providing a blood pressure monitor pneumatically coupled to the blood pressure cuff, providing at least one accelerometer mechanically disposed such that the at least one accelerometer substantially measures a motion of the at least one bladder, attaching the blood pressure cuff to a person, performing an oscillometric procedure using the blood pressure cuff, measuring a pressure and a motion of the at least one bladder during the oscillometric procedure, determining an effect of the motion on the non-invasive blood pressure measurement, and whereby a motion artifact greater than a pre-determined threshold is detected when present during a non-invasive blood pressure measurement.

In one embodiment, the step of determining an effect includes the step of determining an effect of the motion on the non-invasive blood pressure measurement and providing an indication to the person to reduce movement of a limb to which the blood pressure cuff is attached.

In another embodiment, the step of determining an effect includes the step of determining an effect of the motion on the non-invasive blood pressure measurement and indicating to an Operator of the blood pressure monitor to do the non-invasive blood pressure measurement over again where an excessive motion has been detected.

In yet another embodiment, further including, following the step of determining an effect, the step of correcting the non-invasive blood pressure measurement based on the measurement of the motion of the at least one bladder.

### BRIEF DESCRIPTION OF THE DRAWINGS

For a further understanding of these and objects of the invention, reference will be made to the following detailed description of the invention which is to be read in connection with the accompanying drawing, where:

FIG. 1A shows a block diagram of one embodiment of a blood pressure monitor according to the invention.

FIG. 1B shows a block diagram of one embodiment of a pneumatic-mechanical connection between a blood pressure cuff and an electro-pneumatic package of FIG. 1A.

FIG. 1C shows a simplified drawing of one embodiment of a blood pressure monitor according to FIG. 1B.

FIG. 2A shows a flow chart of a blood pressure monitor that compares an accelerometer signal to a threshold value.

FIG. 2B shows a flow chart of a blood pressure monitor that compares saved accelerometer signal values to a threshold value.

FIG. 2C shows a flow chart of a blood pressure monitor that compares accelerometer signal values to pressure signals to determine if there has been significant motion induced artifact error.

FIG. 3A shows a flow chart of a blood pressure monitor that can remove motion induced artifact error and display motion artifact corrected results.

FIG. 3B shows a flow chart of a blood pressure monitor that can remove motion induced artifact error and display motion artifact corrected results and a notice that the results have been corrected for motion artifact.

FIG. 3C shows a flow chart of a blood pressure monitor that can remove motion induced artifact error and display motion artifact corrected results and, when there has been significant motion detected, a notice that the results have been corrected for motion artifact.

FIG. 4A shows a drawing of an exemplary front or top view of a blood pressure monitor.

FIG. 4B shows an exemplary internal view of a pneumatic section of a blood pressure monitor.

FIG. 5A is an illustration of an internal view of an exemplary front or top section of a blood pressure monitor.

FIG. 5B is an illustration of an internal view of an exemplary pneumatic section of a blood pressure monitor.

FIG. 6A shows a drawing of an exemplary internal view of a pneumatic section with a PCB partially removed.

FIG. 6B shows a drawing of an exemplary external view of a pneumatic section including a FLEXIPORT.

FIG. 6C is an illustration of an internal view of an exemplary pneumatic section of a blood pressure monitor with a PCB partially removed to show a more detailed view of the internal pneumatic connections.

The drawings are not necessarily to scale, emphasis instead generally being placed upon illustrating the principles of the invention. In the drawings, like numerals are used to indicate like parts throughout the various views.

### DETAILED DESCRIPTION OF THE INVENTION

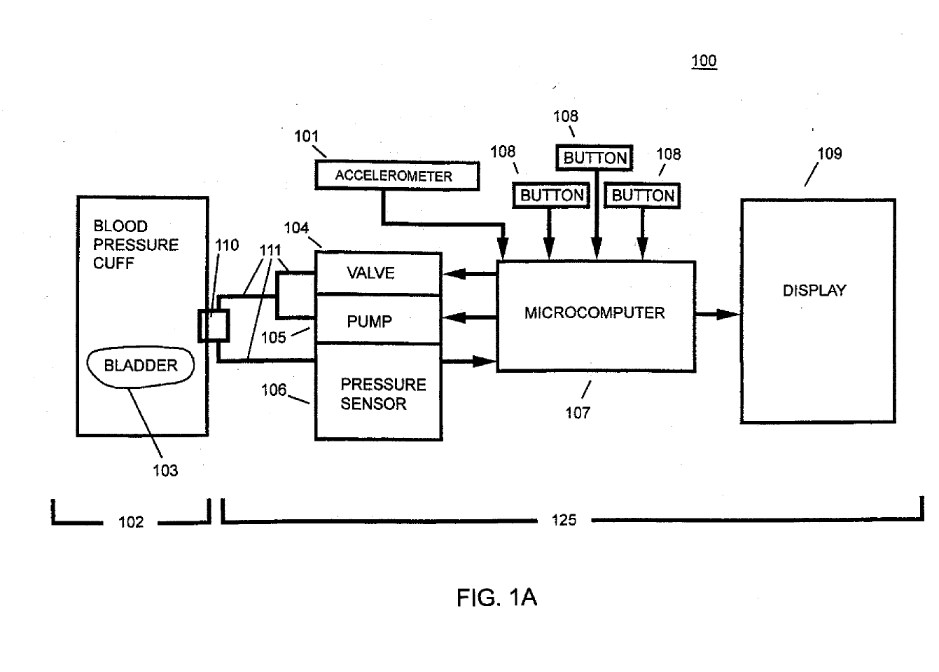

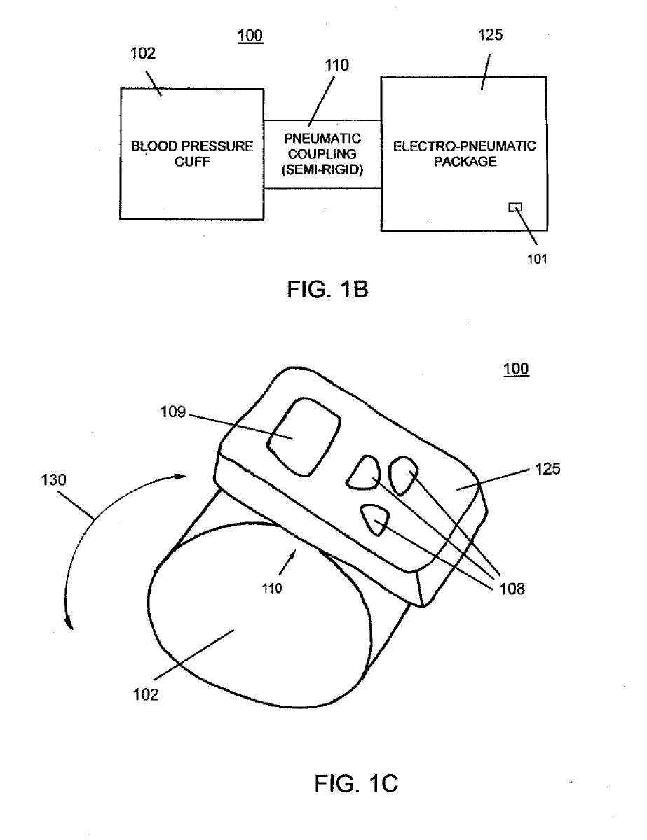

FIG. 1A shows a block diagram of an exemplary embodiment of a blood pressure monitor 100 according to the invention. Accelerometer 101 can be situated on or in an electro-pneumatic package 125 such that it is mechanically coupled to the monitor in such a way that the accelerometer signal substantially reflects any acceleration of blood pressure cuff 102. Blood pressure cuff 102 can include at least one bladder 103. A microcomputer 107 can run an algorithm, typically present as firmware, that controls the various electromechanical components of the blood pressure monitor, processes information, and also typically receives analog and/or digital inputs from electrical signals from sensors, and to display calculated blood pressure information on a display such as display 109.

A blood pressure monitor 100 according to the invention can provide information indicative of a patient's activity level as measured by the one or more accelerometers 101. Such information can be for use solely by the instrument, or transmitted by wired or wireless means to another instrument or computer. Typically a blood pressure monitor as shown in the embodiments of FIG. 1A to FIG. 1C can be used as a stand alone instrument. However, it can also be possible in some more sophisticated embodiments of a blood pressure monitor 100 to transmit information regarding a patient's activity level by wired or wireless means to another medical monitor or directly to a computer or to a computer via a computer network.

Beyond general monitoring of a person's motion or activity, information from the one or more accelerometers 101 can be used by an algorithm running on a microcomputer in blood pressure monitor 100 to discriminate undesired patient motion from the person's heart signal as monitored through pressure measurements of bladder 103. The discrimination feature can be used to enable an alert mechanism for a clinician. In some embodiments, in response to an alert, a clinician can act as needed to settle a patient exhibiting excessive activity. In other embodiments improved performance NIBP algorithms running on a microcomputer, such as microcomputer 107, can ignore or reject pressure signals adversely affected by a person's motion. In still other more sophisticated embodiments, improved performance NIBP algorithms running on a microcomputer, such as microcomputer 107, can cancel patient motion from the corresponding pressure signal used in the blood pressure measurement.

According to the inventive system and method, a blood pressure cuff is first attached to the limb of a person, typically around the person's arm. In a home setting, generally a monitor having a single suitably sized cuff is used. In a clinical setting, there can be a range of cuff sizes available to accommodate different limb sizes and different ages, such as baby, pediatric, and adult. Once attached to the limb, a bladder in a blood pressure cuff can be inflated to a pressure above the highest expected systolic blood pressure, typically to a pressure of on the order of 160 mmHg (1 PSI˜50.17 mmHg). A valve can then be opened and the pressure of the bladder monitored as the bladder deflates. While unimportant to the inventive use of an accelerometer to monitor motion of the bladder, typically in modern blood pressure monitors, a pressure sensor generates an analog or a digital pressure signal over time as the bladder inflates and/or deflates. In the case of such a microcomputer based blood pressure monitor, the blood pressure parameters, such as the systolic pressure, diastolic pressure, and mean arterial pressure, can then be calculated from the pressure data and displayed, such as on a LCD display.

We define the term “oscillometric procedure” herein as including known techniques and algorithms for measuring blood pressure using, for example, a blood pressure cuff having at least one inflatable bladder. It is unimportant for the inventive technique or associated apparatus whether the bladder pressure measurements used to determine the desired blood pressure parameters, are taken, recorded, digitized, or measured during inflation, deflation, or during a combination of inflation and deflation. For example, one useful oscillometric technique, the Welch Allyn FastBP® system, measures blood pressure cuff bladder pressure during inflation. U.S. patent application Ser. No. 11/347,889, published as U.S. Published Patent Application No. 2007/0185401, “Blood Pressure Measurement”, describes one such oscillometric technique. The '889 application is incorporated herein by reference in its entirety.

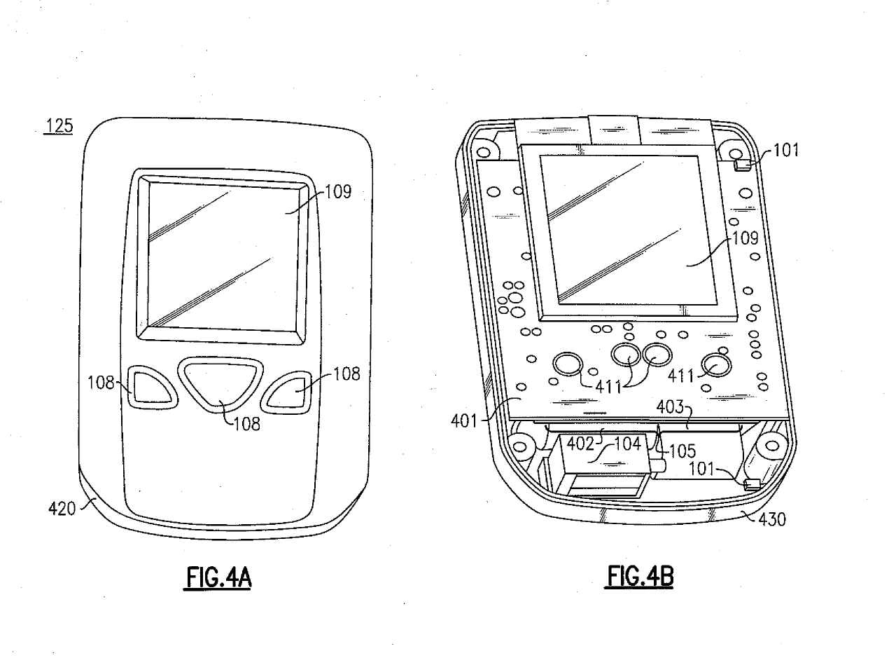

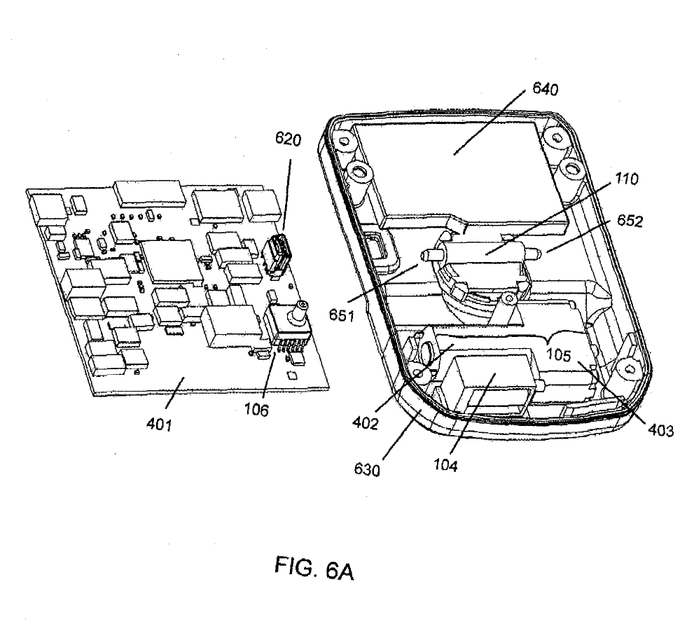

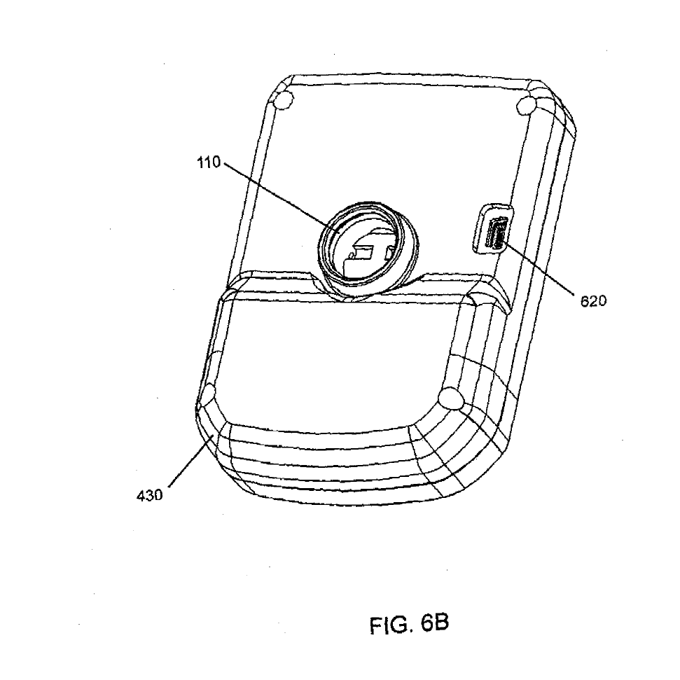

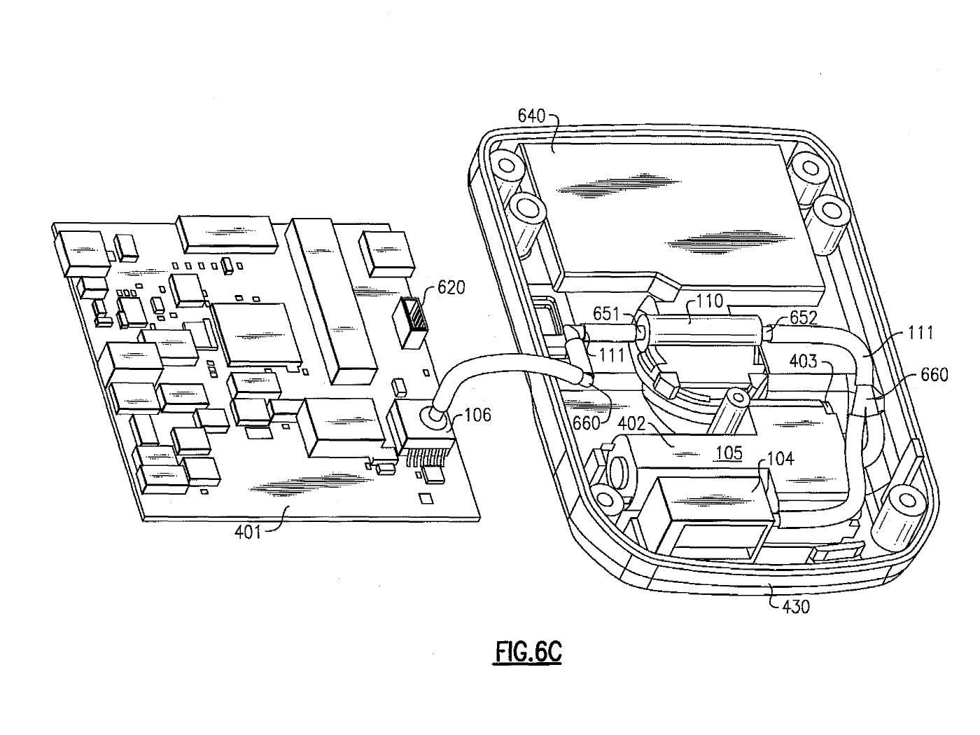

Returning now to FIG. 1A, we describe a blood pressure monitor 100 in more detail. The electromechanical components can include a valve 104, a pump 105, and a pressure sensor 106. User accessible buttons 108 can allow for user interaction with the blood pressure monitor. Such interaction can include initiation of a blood pressure reading, setting date and time, and selecting modes of operation. In the block diagram of FIG. 1A, it understood that at least one source of power, typically one or more batteries not shown for simplicity, are present. Also not shown are the interface electronics blocks, such as transistor switches, typically FET switches, that allow a microcomputer 107 to drive relatively high current loads such as a valve 104, typically a solenoid operated valve, and a pump 105, typically including a pump motor 402 and pneumatic pump head 403 (FIG. 4A, FIG. 6B). The drive power for valves and pumps can be simple DC switching, such as using ON-OFF solid state switches, or can by pulse width modulation (PWM). PWM drive can be particularly useful for driving a pump motor and can include such features as a pump motor start profile in time. It is also understood that signals from electronic sensors, such as pressure sensor 106 and accelerometer 101 can be analog or digital signals. Typically such sensors have analog outputs, such as analog voltage outputs. The analog voltages outputs can be signal conditioned, typically including low pass anti-aliasing filtering, and digitized, such as by an analog to digital converter (ADC). The ADC, not shown in the simplified block diagram of FIG. 1A can be separate from microcomputer 107 or included within microcomputer 107. There can be more than one ADC, however, more typically one ADC having multiple analog signal inputs can be used, such as by using a multiplexed or multiple input ADC external to microcomputer 107 or within microcomputer 107.

FIG. 1B shows a simplified block diagram of an embodiment of the blood pressure monitor of FIG. 1A where the blood pressure cuff 102 can be mechanically connected to electro-pneumatic package 125 by a pneumatic coupling 110. Pneumatic coupling 110 can provide a semi-rigid, yet substantially air-tight, mechanical coupling between blood pressure cuff 102 can be mechanically connected to electro-pneumatic package 125. Pneumatic coupling 110 can be a “FLEXIPORT” pneumatic coupling. A FLEXIPORT pneumatic coupling 110 is described below in more detail.

FIG. 1C is a representative drawing showing one embodiment of a blood pressure monitor 100 according to the simplified block diagram of FIG. 1B. As can be seen in FIG. 1C, in this exemplary embodiment, there is a direct coupling between electro-pneumatic package 125 and blood pressure cuff 102, that is the entire pneumatic coupling can be provided by pneumatic coupling 110 without need for any additional pneumatic hose or pipe between electro-pneumatic package 125 and blood pressure cuff 102. It is understood, however, that pneumatic hose 111 can be used to complete the “pneumatic circuit” within electro-pneumatic package 125 as is shown in FIG. 1A and discussed in the example as illustrated by FIG. 6C.

Here it can be seen that in some embodiments, a semi-rigid pneumatic coupling 110 can be configured to provide rotation or swivel feature such that when blood pressure cuff 102 is affixed on the limb of a person, electro-pneumatic package 125 can be rotated 130 to a convenient operating and viewing angle. While pneumatic coupling 110 is not visible in FIG. 1B, an arrow indicates the general location between electro-pneumatic package 125 and blood pressure cuff 102. It can also be better seen in FIG. 1C how a mechanical acceleration of blood pressure cuff 102 can be mechanically transmitted through a rigid or semi-rigid pneumatic coupling 110 to one or more accelerometers 101 (not shown in FIG. 1C) disposed in or on an electro-pneumatic package 125. Accelerometer 101 can be attached to any part of the monitor apparatus that can mechanically transmit motion of bladder 103 to accelerometer 101, including on or in a pneumatic coupling 110, however, in most embodiments one or more accelerometers 101 can be disposed on or in electro-pneumatic package 125. One advantage of situating the one or more accelerometers 101 on or in electro-pneumatic package 125 is cost savings in manufacture, since it is relatively easy to add electronic components to electro-pneumatic package 125 where there is already substantial electronics present. Another advantage is that compatible blood pressure cuffs with a suitable mating pneumatic coupling 1 10 do not need the additional cost or complexity of one or more accelerometers 101, associated wiring and electrical connectors. It is only important that an electrical signal derived from accelerometer 101 substantially indicate mechanical motion or movement of the bladder 103.

In the most basic embodiments, it might only be possible to include a motion warning indication, such as by a warning light visible display indication, or audible sound or some combination of audio and/or visual warnings. In embodiments having more sophisticated electronics, there can be a display indicating through icons and/or text that there is excessive motion of a person's limb (patient activity) to which a blood pressure cuff 102 is affixed. In still more sophisticated embodiments using electronics packages including microcomputers, such as the apparatus illustrated in FIG. 1A to FIG. 1C, there can be embodiments that also substantially cancel the effects of motion by applying a correction signal from an accelerometer to the pressure signal from a pressure sensor 106 to cancel, or to remove at least in part, the adverse effect of the measured motion the pressure signal.

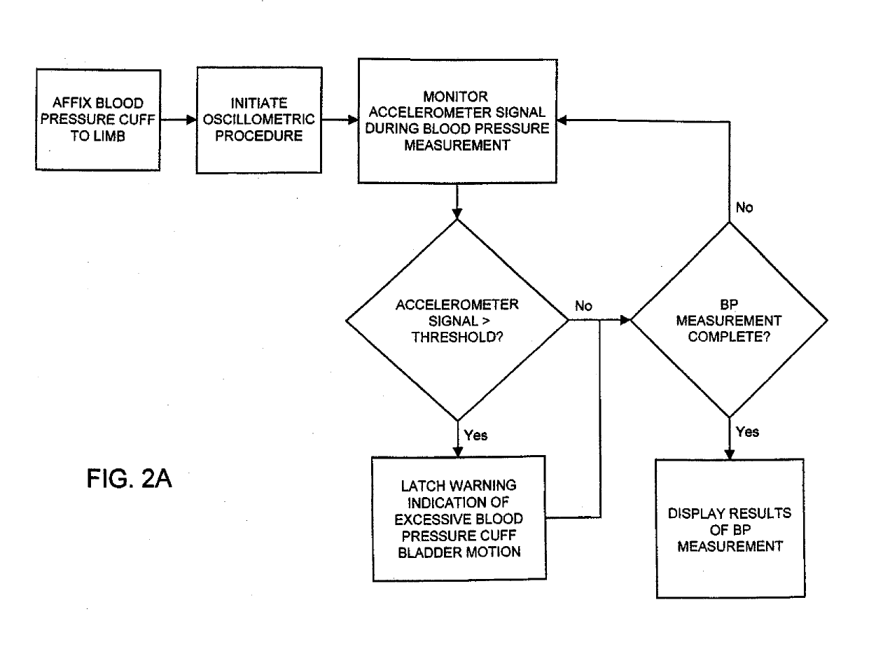

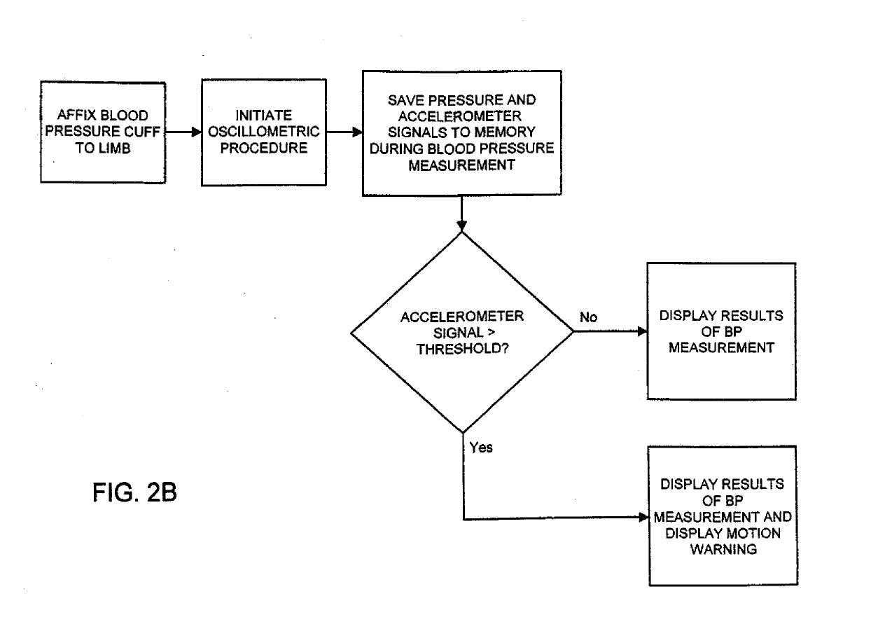

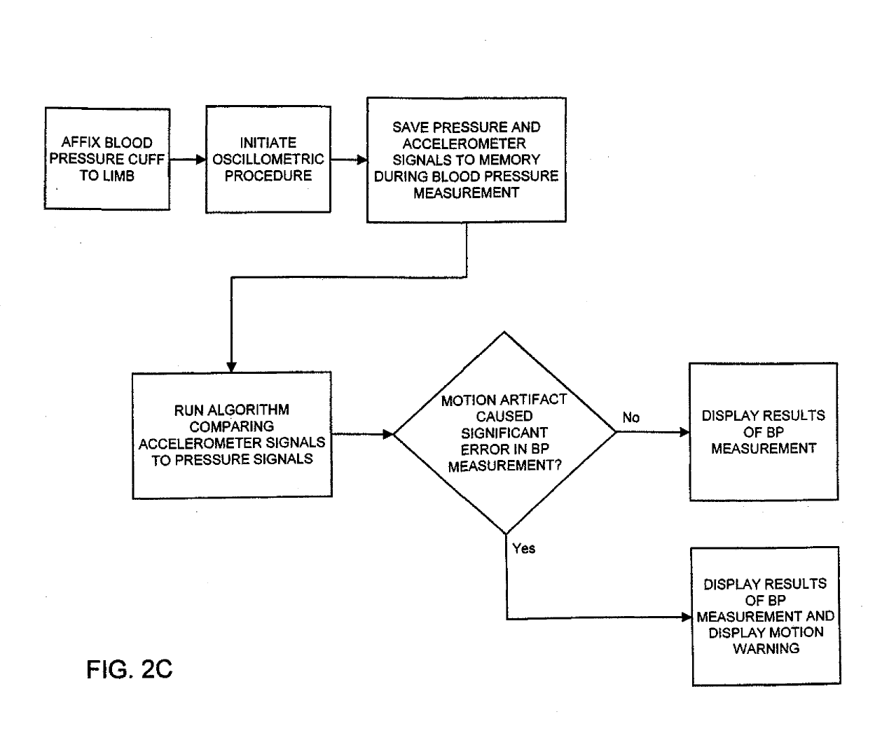

In the exemplary flow charts that follow, we describe various embodiments of such NIBP algorithms in more detail. Turning to the flow charts of FIG. 2A, FIG. 2B, and FIG. 2C, we now describe in more detail the operation one embodiment of a blood pressure monitor 100 using an accelerometer 101. In these embodiments, an accelerometer serves to warn the operator of a blood pressure monitor that there is excessive movement of a person's limb to which a blood pressure cuff 102 has been affixed. The operator and the person can be one in the same, as in a person monitoring their own blood pressure, or different persons, such as where a clinician is measuring the blood pressure of a patient.

In one embodiment of an inventive blood pressure monitor, as illustrated by the flow chart of FIG. 2A, an analog or digital acceleration signal representing motion of a bladder in the blood pressure cuff can be continuously sampled and compared to at least one pre-set threshold level. A visual or audio indication can be conveyed to the operator if the motion of the bladder, as represented by the acceleration signal, exceeds the preset threshold. The indication can be a visual indicator or audio warning or text on a display such as “There was excessive motion, please repeat the measurement.” The visual or audio indication can also be “latched” on as shown in the exemplary flow chart of FIG. 2A, so the warning can be present until reset or until another blood pressure measurement is commenced, such as by manual operation of a start or measure button.

Another embodiment, similar to that illustrated in FIG. 2A, is illustrated by the flow chart of FIG. 2B. The difference is that in the embodiment of FIG. 2B, the acceleration signal can be digitized and saved to a memory, typically at intervals during or near the time that pressure sensor measurements are recorded during the bladder deflation. The memory can either be situated within a microcomputer or connected to a microcomputer. After the bladder deflation is complete, an algorithm running on the microcomputer can evaluate the recorded accelerometer signal over time and the algorithm can make a determination if the recorded motion of the bladder during deflation adversely effected the blood pressure measurement. In a most basic version of the embodiment of FIG. 2B, the acceleration signal can be compared to a pre-determined threshold value. If any points, or a mathematical combination of points, such as a running box-car average, exceeds the threshold, an indication can be given to the operator that there was excessive motion during the measurement. The indication can be a visual or audio warning or text on a display such as “there was excessive motion”, please repeat the measurement. In a more sophisticated embodiment as illustrated by the exemplary flow chart of FIG. 2C, there can be an analysis of the acceleration signal in time as compared to a bladder pressure signal in time. A slightly more sophisticated algorithm can make a determination if the motion occurred at a time and/or was of a level to significantly adversely effect the blood pressure measurement to an extent that the operator should be notified of motion artifact induced error and to repeat the measurement.

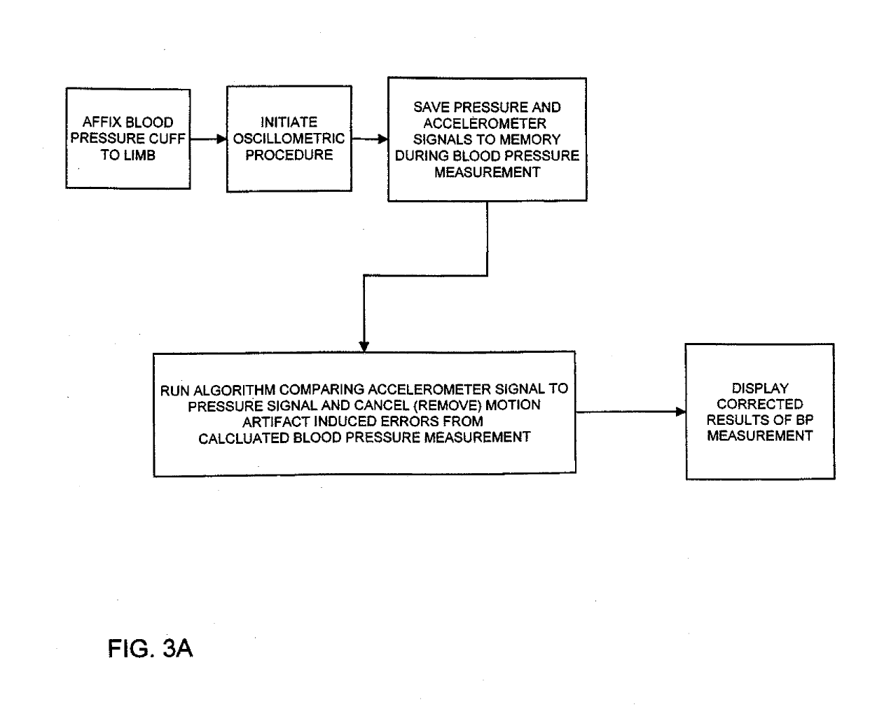

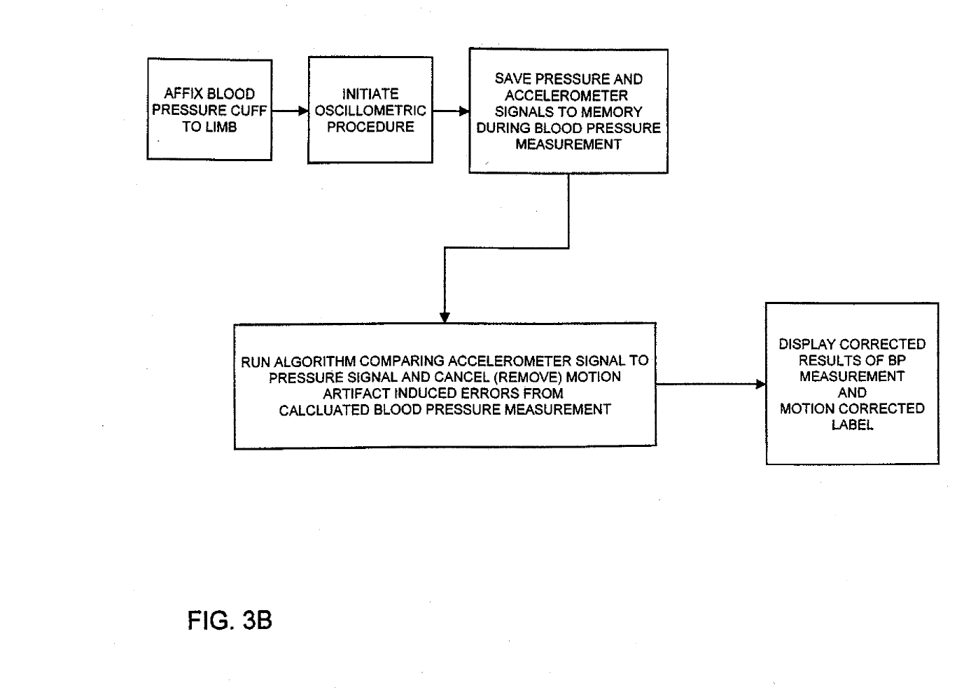

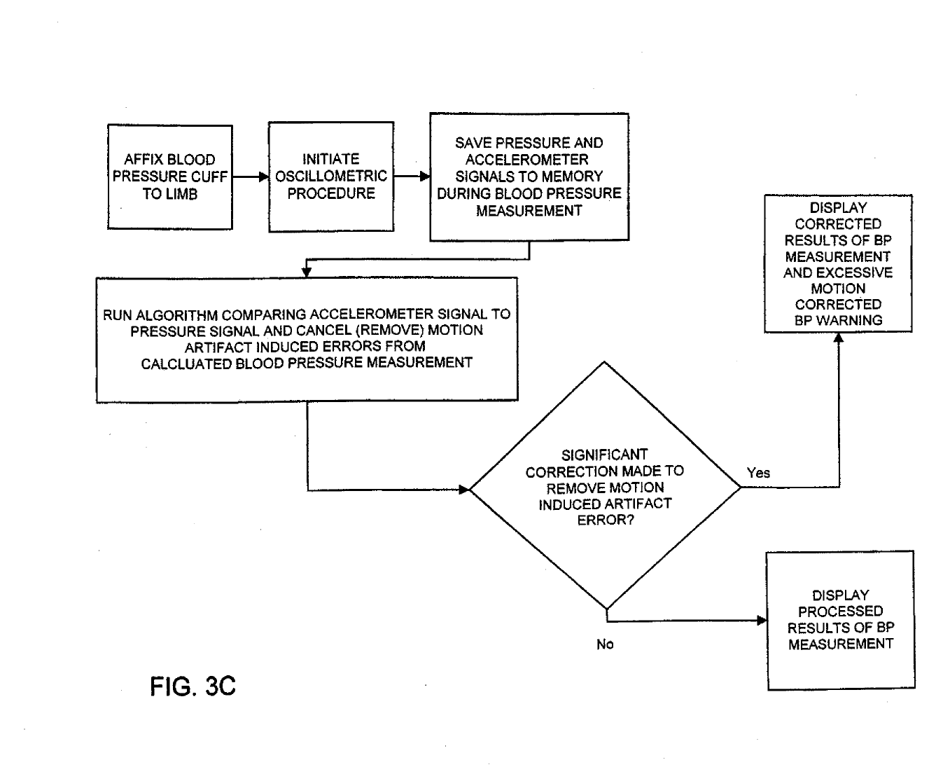

In the embodiment illustrated by the exemplary flow chart of FIG. 3A, accelerometer measurements can be saved to memory during or near the time that pressure measurements are also being saved to memory. An algorithm running on a microcomputer can include the accelerometer measurements in the blood pressure parameter calculations in order to substantially cancel or remove the effects of bladder motion from the pressure measurements. Typically mathematical scaling including offset and scale factors can be used to make a first order accelerometer correction. More accurate cancellation may be achieved using non-linear mathematical operations involving either the accelerometer measurement data or the pressure data or both. In the embodiment of FIG. 3A, only the resultant blood pressure measurements are displayed and the entire acceleration correction process is entirely transparent to a user of the blood pressure monitor. The exemplary embodiment illustrated by the flow chart of FIG. 3B is the same as that shown in FIG. 3A, except that the reading can also be labeled as having been corrected for bladder or blood pressure cuff motion. The exemplary embodiment illustrated by the flow chart of FIG. 3C is the same as that shown in FIG. 3B, except that where there is a certain level of correction, such as motion beyond a certain threshold level, the display can include the corrected blood pressure measurements in addition to a displayed warning that there was significant motion. In the embodiment of FIG. 3C, where excessive motion is not detected, the display can show the standard or requested blood pressure parameters, without reference to motion or motion corrected readings.

example: We now describe a compact wearable blood pressure monitor according to the invention. The wearable blood pressure monitor includes both an electronics/pneumatic package (the NIBP monitor) and a blood pressure cuff having an inflatable bladder. In the exemplary blood pressure monitor, the blood pressure cuff attaches directly to the electronics/pneumatic package by a FLEXIPORT (a substantially rigid mechanical and pneumatic connection of the cuff to the NIBP monitor). A FLEXIPORT connection is described in more detail in two related U.S. patent applications Ser. No. 11/230,117, entitled Blood Pressure Measuring Apparatus”, and U.S. patent applications Ser. No. 11/513,608, entitled “Blood Pressure Measuring Apparatus”, both applications also assigned to the Welch Allyn Corporation. The Ser. No. 11/230,117 and the Ser. No. 11/513,608 applications are both incorporated herein by reference in their entirety. In addition, to accomplish the inventive technique, one or more accelerometers are mounted on or in the electronics/pneumatic package. The accelerometer can be mounted on a printed circuit board (PCB), elsewhere in or on the enclosure, or in or on the FLEXIPORT. Since the electronics/pneumatic package is mechanically coupled to the blood pressure cuff by the relatively rigid FLEXIPORT connection, any motion of the blood pressure cuff is substantially transmitted to the accelerometer disposed in or on the electronics/pneumatic package or in or on the FLEXIPORT.

In the exemplary NIBP monitor, the blood pressure cuff need only be FLEXIPORT compatible, otherwise the cuff itself does not need to include an accelerometer. One advantage of system a NIBP monitor is that there need only be one version of FLEXIPORT blood pressure cuffs, albeit in various sizes where multiple persons having different limb sizes are monitored. Also, since in this example the accelerometer is not disposed in the blood pressure cuff, there is no need for electrical connections between the FLEXIPORT compatible cuff and the FLEXIPORT connection or port.

FIG. 4A and FIG. 4B show one exemplary open view of a blood pressure monitor 100 according to the invention. FIG. 4A is an exemplary view of a section 420, typically a front or top section, that can be mounted to an exemplary electro-pneumatic package 430. Section 420 can include buttons 108 and display 109 for interaction with a user/operator. A section 430 is typically closely coupled to a blood pressure cuff (not shown in FIG. 4A), such as by affixing the electro-pneumatic package 430 directly to a blood pressure cuff. The larger button 108 can serve as a “measure” button. Two or more sets of conductive traces 411, or other suitable types of button mechanisms, can be co-located to enhance ease of use and/or reliability for the most frequently used buttons 108. Smaller buttons 108 can be used to select modes of operation and to perform housekeeping functions such as to set the current date and time. Display 109 of the example can be seen as displaying a systolic pressure (120) over a diastolic pressure (80), a MAP reading (93), and a heart rate (60). Below the pressure readings, and not clearly visible in the example, can be a date and time display and any motion warning or motion correction display text as previously described with regard to the exemplary flow charts.

FIG. 4B shows an internal view of the exemplary electro-pneumatic package 430, typically present as a bottom or back section that can be mechanically joined to a section 420. Accelerometer 101 is shown in two exemplary alternate locations, on printed circuit board 401 or mounted elsewhere in the electro-pneumatic package 430 not directly on printed circuit board 401. Typically only one accelerometer 101, such as three axis accelerometer, would be present, such as disposed in or on a blood pressure monitor housing. Since the blood pressure cuff 102 (not shown in FIG. 4A or FIG. 4B) can be affixed directly to the exemplary electro-pneumatic package 430, any motion registered by an accelerometer 101 disposed in the electro-pneumatic package 430 will be substantially representative of motion of the attached blood pressure cuff 102 and therefore also of at least one bladder 103 disposed in the blood pressure cuff 102.

While in the exemplary embodiment of FIG. 4A and FIG. 4B section 420 includes user interface components and electro-pneumatic package 430 contains most of the related electronics and pneumatic components, it is unimportant to the invention which components are packaged in which section. The number of sections or housings is also unimportant. The components of the inventive blood pressure monitor can be included in one enclosure not separable into sections or in more than two sections.

A portion of pump 105 including a motor (cylindrical portion) and pump head (adjacent to the motor) can also be seen FIG. 41. Pump 105 is partially obscured by printed circuit board 401. Valve 104 is symbolically represented. Pneumatic connections, typically a pneumatic coupling or plastic tubes suitable for pneumatic use, are understood to be pneumatically coupled between the valve 104, pump 105 and bladder 103 (not shown in FIG. 4B). One exemplary pneumatic coupling particularly well suited for use in such a blood pressure monitor is a FLEXIPORT.

Display 109 can be seen represented as an exemplary LCD display and can be of various useable resolutions and monochromatic such as black or blue or a color display. A symbolic power source can be seen to be represented by battery 410 including for example, one or more button batteries. A pressure sensor 106 (not shown in FIG. 4B) is also understood to be present. A pressure sensor such as a model no. XFPM-050 manufactured by Fujikura Ltd. of Tokyo, Japan, is suitable for use as pressure sensor 106.

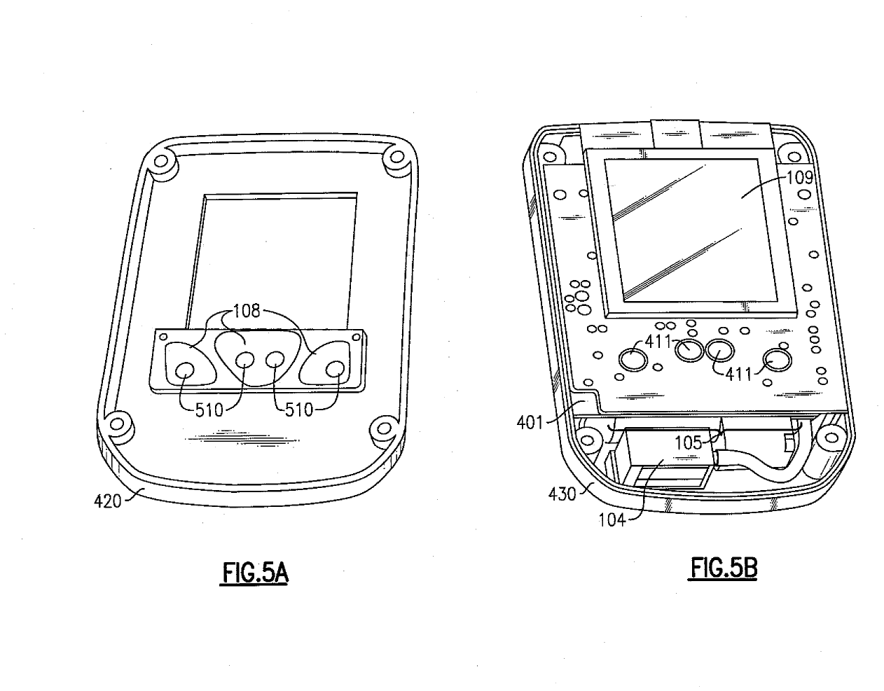

FIG. 5A and FIG. 5B show illustrations of an opened blood pressure including a section 420 and an electro-pneumatic package 430 suitable for use with the inventive motion sensing technique. The inside view of buttons 108 can also be seen in this exemplary embodiment to include conductive pads 510. As can be seen in the corresponding FIG. 5B, when buttons 108 are depressed by a user from the outside surface of section 420, conductive pads 510 come in physical and electrical contact with conductive traces 411 on PCB 401 so as to cause an electrical indication of button 108 operation.

FIG. 6A shows a drawing of an exemplary embodiment of a blood pressure monitor similar to that shown in the illustration of FIG. SA and FIG. 5B. Here, PCB 401 is shown removed from an electro-pneumatic package 430 with a component side exposed. One suitable location for pressure sensor 106 is shown in FIG. 6A as mounted to PCB 401. While it is not important to mount a component side of PCB 401 facing into electro-pneumatic package 430, it can be seen that this approach can facilitate the pneumatic connection to some models of pressure sensor 106. Also, in the disassembled view of electro-pneumatic package 430, a view of a FLEXIPORT 110 can be seen as including pneumatic connections 651 and 652 where pneumatic connections 651 and 652 typically accept a pneumatic hose such as a plastic, nylon, vinyl, polyvinyl, polystyrene, or similar suitable pneumatic hose. Also visible in FIG. 6A is a block representing one practical location for a battery 640. An optional USB connector 620 is also visible on PCB 401.

FIG. 6B shows a drawing of the exemplary embodiment of a blood pressure monitor of FIG. 6A. The view of FIG. 6B, typically a rear or back view, is of a side of an electro-pneumatic package 430 having a FLEXIPORT 11 0 that can in some embodiments, directly pneumatically couple to a blood pressure cuff 102. USB connector 620 can also be seen as creating a user/operator accessible USB port for connecting a computer via a USB interface as can be present on PCB 401 (not shown in FIG. 6B). Such connections can be used to upload or download blood pressure measurements, data sets, configurations, and for making firmware upgrades to the blood pressure monitor 100.

FIG. 6C shows an illustration of one embodiment of an actual blood pressure monitor suitable to accept an accelerometer for performing the inventive technique. FIG. 6C shows in more detail the pneumatic connections of electro-pneumatic package 430, including pneumatic hoses 111 and pneumatic couplings 660.

Accelerometer 101 can be any type of accelerometer suitable for use to detect motion of a bladder 103 of a blood pressure cuff 102. Typical suitable accelerometers include solid state accelerometers such as those using MEMS technologies. MEMS accelerometers are available from a number of companies including: Freescale Semiconductor of Austin, Tex., Analog Devices or Norwood, Mass., Infineon Technologies of Los Angeles, Calif., Memsic of Andover, Mass. and China, Bosch Sensortec of Reutlingen, Germany, Hitachi Metals of Tokyo, Japan, Oki Electric Industries of Tokyo, Japan, and Kionix of Ithaca, N.Y. While it may be possible to detect blood pressure cuff motion using an accelerometer having one or two sensitive axis to successfully, full three axis (x,y,z) sensitivity can be preferable. It is unimportant to the invention whether three separate analog or digital signals are received from an accelerometer 101 and further processed, such as to generate a motion vector having a magnitude and/or direction, or if the output from an accelerometer 101 is a single analog or digital magnitude and/or direction. It is also unimportant whether a single multi-axis accelerometer or two or more single axis accelerometers are mechanically disposed to be sensitive in two or more axis.

A person's or patient's activity level as measured by the one or more accelerometers is defined as a physical motion of some part of a person's body where that motion transmits to the part of the person's body to which a blood pressure cuff is affixed, typically a person's limb. The motion can be in another part of the body, such as the torso and mechanically transmitted to the relevant limb, or the motion can be caused by motion of the limb itself. Such a motion can result in a motion artifact error in a blood pressure measurement, typically by causing an acceleration of a bladder in a blood pressure cuff. The acceleration of the cuff can cause a pressure signal related to the motion that can distort the pressure reading (pressure sensor signal) from the blood pressure cuff that otherwise could produce an accurate measurement of the person's blood pressure. We define such errors interchangeably herein by the terms and phrases including, but not limited to, motion error, motion induced error, motion artifact error, excessive motion error, and motion induced artifact error.

We define “microcomputer” herein as synonymous with microprocessor, microcontroller, and digital signal processor (“DSP”). It is understood that memory used by the microcomputer, including for example blood pressure monitor “firmware”, can reside in memory physically inside of a microcomputer chip or in memory external to the microcomputer or in a combination of internal and external memory. Similarly, analog signals can be digitized by one or more stand alone analog to digital converter (“ADC”) or one or more ADCs or multiplexed ADC channels can reside within a microcomputer package. It is also understood that field programmable array (“FPGA”) chips or application specific integrated circuits (“ASIC”) chips can perform microcomputer functions, either in hardware logic, software emulation of a microcomputer, or by a combination of the two. Blood pressure monitors having any of the inventive features described herein can operate entirely on one microcomputer or can include more than one microcomputer.

A wireless connection made by a blood pressure monitor 100 can be 802.11 compliant, or can use a lighter-weight (simpler) protocol that can be more energy efficient. A suitable lighter weight protocol can be proprietary, or standards-based, such as ZigBee or Bluetooth. A blood pressure monitor 100 having wireless capability can be used in hospital environment as part of an integrated wireless monitoring network. The details of such monitoring networks are disclosed in U.S. patent application Ser. No. 11/031,736 entitled, “Personal Status Physiological Monitor System and Architecture and Related Monitoring Methods”, which is incorporated by reference herein in its entirety.

We claim:

1. A blood pressure monitor for measuring the blood pressure of a person comprising:
a blood pressure cuff, said blood pressure cuff comprising at least one bladder;
an electro-pneumatic package, said electro-pneumatic package including a pump, a valve, a pressure sensor, and one or more accelerometers;
a pneumatic mechanical coupling, said pneumatic mechanical coupling configured to pneumatically and mechanically directly couple said blood pressure cuff to said electro-pneumatic package, wherein a signal from said one or more accelerometers configured to indicate an activity level of said person during a blood pressure measurement; and
a display, said display configured to display an indication of said activity level.

2. The blood pressure monitor of claim 1, wherein said signal from said one or more accelerometers indicates an activity level of said person during a blood pressure measurement and wherein an algorithm running on a microcomputer is configured to receive one or more values representative of said signal from said one or more accelerometers and said algorithm rejects at least one pressure value representative of a pressure signal from said pressure sensor as cause to be in error by said activity level of said person.

3. The blood pressure monitor of claim 1, wherein said signal from said one or more accelerometers indicates an activity level of said person during a blood pressure measurement and wherein an algorithm running on a microcomputer is configured to receive one or more values representative of said signal from said one or more accelerometers and said algorithm corrects at least one pressure value representative of a pressure signal from said pressure sensor to substantially remove motion induced error caused by said activity level of said person.

4. The blood pressure monitor of claim 1, wherein said signal from said one or more accelerometers indicates an activity level of said person during a blood pressure measurement, said activity level of said person causing a motion induced error, and wherein an algorithm running on a microcomputer is configured to receive one or more values representative of said signal from said one or more accelerometers and said algorithm corrects the blood pressure of said person to substantially remove said motion induced error.

5. A blood pressure monitor comprising:
a blood pressure cuff including at least one bladder, said blood pressure cuff also including a first half of a mechanical pneumatic connector;
a pump, said pump pneumatically coupled to said bladder, said pump configured to inflate said bladder;
a valve, said valve pneumatically coupled to said bladder, said valve configured to cause a controlled deflation of said bladder;
a pressure sensor, said pressure sensor pneumatically coupled to said bladder, said pressure sensor configured to measure a bladder pressure;
one or more accelerometers, said one or more accelerometers electrically and mechanically configured to provide a motion signal from said one or more accelerometers responsive to and representative of a movement of said at least one blood pressure inflatable bladder; and
an electronics circuit, said electronics circuit electronically coupled to said valve, said pump, said pressure sensor, and said one or more accelerometers,
wherein said pump, said valve, said pressure sensor, said one or more accelerometers, and said electronics circuit are disposed within an electro-pneumatic package, and said electro-pneumatic package comprises a second half of a mechanical pneumatic connector and wherein said first half of said mechanical pneumatic connector is configured to mechanically connect to said second half of a mechanical pneumatic connector to provide a substantially air-tight semi-rigid mechanical and pneumatic direct coupling between said electro-pneumatic package and said blood pressure cuff and wherein said electronics circuit is configured to receive said motion signal from said one or more accelerometers during a blood pressure measurement.

6. The blood pressure monitor of claim 5, wherein said electronics circuit is configured to cause an indication of excessive motion based on said motion signal from said one or more accelerometers representative of said movement of said at least one bladder when said motion signal exceeds a pre-determined threshold.

7. The blood pressure monitor of claim 6, wherein said indication of excessive motion comprises one of a visual indication and an audio indication.

8. The blood pressure monitor of claim 6, wherein said indication of excessive motion is configured to indicate that said blood pressure measurement should be repeated.

9. The blood pressure monitor of claim 5, wherein said electronics circuit further comprises a microcomputer, said microcomputer configured to receive said motion signal from said one or more accelerometers and to correct said blood pressure measurement based on said motion signal.

10. The blood pressure monitor of claim 5, wherein said one or more accelerometers comprise a MEMS accelerometer.

11. The blood pressure monitor of claim 10, wherein said MEMS accelerometer comprises a three axis accelerometer.

12. The blood pressure monitor of claim 5, wherein said at least one accelerometer is mechanically disposed on or in a blood pressure monitor housing.

13. The blood pressure monitor of claim 5 wherein said motion signal comprises one or more analog signals from said one or more accelerometers and wherein said indication of excessive motion is based at least in part upon said motion signal and said motion signal threshold.

14. The blood pressure monitor of claim 5 wherein said motion signal comprises one or more analog signals from said one or more accelerometers and said electronics circuit comprises at least one analog to digital converter (ADC), said at least one ADC configured to digitize said one or more analog signals from said one or more accelerometers, and wherein said indication of excessive motion is based at least in part upon said motion signal in a digital form and said motion signal threshold in a digital form.

15. The blood pressure monitor of claim 5 wherein said electronics circuit is additionally configured to provide a correction to substantially correct said blood pressure measurement for a blood pressure cuff motion induced error.

16. The blood pressure monitor of claim 15 wherein said correction to said blood pressure measurement comprises a correction based on analog signals or a correction based on digital signals.

17. The blood pressure monitor of claim 15 wherein said correction is configured to be applied to a digital representation of a pressure signal from said pressure sensor.

18. The blood pressure monitor of claim 15 wherein said correction is configured to be applied as part of a digital computation used to calculate said blood pressure measurement.

19. A method for detecting a motion artifact in a non-invasive blood pressure measurement comprising the steps of:
providing a blood pressure cuff having at least one bladder;
providing a blood pressure monitor pneumatically coupled to said blood pressure cuff;
providing at least one accelerometer mechanically disposed such that said at least one accelerometer substantially measures a motion of said at least one bladder;
attaching said blood pressure cuff to a person;
performing an oscillometric procedure using said blood pressure cuff;
measuring a pressure and a motion of said at least one bladder during said oscillometric procedure;
determining an effect of said motion on said non-invasive blood pressure measurement; and
whereby a motion artifact greater than a pre-determined threshold is detected when present during a non-invasive blood pressure measurement.

20. The method of claim 19, wherein said step of determining an effect comprises the step of determining an effect of said motion on said non-invasive blood pressure measurement and providing an indication to said person to reduce movement of a limb to which said blood pressure cuff is attached.

21. The method of claim 19, wherein said step of determining an effect comprises the step of determining an effect of said motion on said non-invasive blood pressure measurement and indicating to an operator of said blood pressure monitor to do said non-invasive blood pressure measurement over again where an excessive motion has been detected.

22. The method of claim 19, further comprising, following the step of determining an effect, the step of correcting said non-invasive blood pressure measurement based on the measurement of said motion of said at least one bladder.
